(19) United States
(12) Patent Application Publication
(10) Pub. No.: US 2025/0179134 A1
(43) Pub. Date: Jun. 5, 2025

(54) METHODS AND COMPOSITIONS FOR TREATING OR AMELIORATING CARDIAC MUSCLE ARRHYTHMIAS AND SKELETAL MUSCLE TREMORS

(71) Applicant: ARIZONA BOARD OF REGENTS ON BEHALF OF THE UNIVERSITY OF ARIZONA, Tucson, AZ (US)

(72) Inventor: Samantha P. Harris, Tucson, AZ (US)

(21) Appl. No.: 18/725,492

(22) PCT Filed: Feb. 20, 2023
(86) PCT No.: PCT/US2023/062906

§ 371 Date: Jun. 28, 2024

Related U.S. Application Data
(60) Provisional application No. 63/311,608, filed on Feb. 18, 2022.

(51) Int. Cl. C07K 14/47

(52) U.S. Cl. CPC C07K 14/4716

(57) ABSTRACT

Methods and compositions for modulating, e.g., damping, contractile oscillations by targeting and/or mimicking My BP-C in heart and skeletal muscles, including therapeutics (e.g., small molecules, peptides, enzymes, antibodies, oligonucleotides, etc.) designed to directly target contractile oscillations and/or MyBP-C. The present invention also includes methods of treating cardiac arrhythmias and/or skeletal muscle tremors by modulating/damping contractile oscillations. e.g., by targeting and/or mimicking My BP-C in the appropriate muscles (e.g., heart, skeletal muscle).

### CROSS-REFERENCES TO RELATED APPLICATIONS

This application claims benefit of U.S. Provisional Application No. 63/311,608 filed Feb. 18, 2022, the specification of which is incorporated herein in their entirety by reference.

### STATEMENT REGARDING FEDERALLY SPONSORED RESEARCH OR DEVELOPMENT

This invention was made with government support under Grant Nos. HL080367 and HL140925 awarded by National Institutes of Health. The government has certain rights in the invention.

### BACKGROUND OF THE INVENTION

### Field of the Invention

The present invention relates to muscle contraction and abnormalities such as but not limited to cardiac arrhythmias and skeletal muscle tremors, more particularly to methods and compositions for treating cardiac arrhythmias and skeletal muscle tremors or any other conditions associated with contractile oscillations.

### Background Art

Efficient cell-to-cell communication is essential to generate coordinated heartbeats that allow the heart to eject blood to the body during each heartbeat forcefully. Coordination occurs partly because of a tightly controlled process where the electrical excitation of each cardiomyocyte signals contraction and relaxation in a synchronized manner. Cardiac arrhythmias, such as those that cause atrial fibrillation or sudden cardiac death (SCD), are typically viewed as electrical/conduction abnormalities that precede (e.g., drive) uncoordinated heartbeats. Accordingly, most antiarrhythmic drugs are ion channel blockers that alter electrical conduction or cell calcium signaling pathways by affecting excitation-contraction coupling where electrical/chemical signaling occurs upstream of muscle contraction.

It was surprisingly discovered that abnormalities within the sarcomeres by themselves potentially can directly initiate uncoordinated heartbeats in cardiac muscle and/or cause tremors in skeletal muscle via mechanical oscillations (e.g., cycles of contraction and relaxation) that are intrinsic properties of muscle sarcomeres. Mechanical oscillations are, as of yet, poorly defined but are observed as cycles of contraction and relaxation in sarcomeres that occur without concomitant changes in cytosolic calcium concentrations that ordinarily drive muscle contraction and relaxation. Without wishing to limit the present invention to any theory or mechanism, it is believed that sarcomere contractile oscillations can directly cause arrhythmias and/or muscle tremors via a contraction-excitation coupling, and sarcomeres can initiate uncoordinated contractions in the absence of aberrations in upstream signaling pathways.

### BRIEF SUMMARY OF THE INVENTION

The present invention provides methods and compositions for modulating, e.g., damping, contractile oscillations. As an example, the present invention provides methods and compositions for modulating, e.g., damping, contractile oscillations by targeting and/or mimicking myosin binding protein C (MyBP-C) in muscles, e.g., heart and skeletal muscles. The present invention includes therapeutics (e.g., small molecules, peptides, enzymes, antibodies, oligonucleotides, drugs, compositions, physical stimuli, etc.) that regulate, e.g., dampen the oscillations. In certain embodiments, the therapeutics target MyBP-C or cMyBP-C. However, the present invention is not limited to targeting MyBP-C or cMyBP-C and includes targeting other molecules involved in the contractile oscillation process. As a non-limiting example, it may be determined that myosin is an appropriate target; other targets may be found to include protein kinase A (PKA) or other signaling molecules involved in MyBP-C's (or cMyBP-C) function, molecules or pathways involved in muscle lattice spacing, etc.

The present invention also includes methods of treating cardiac arrhythmias and/or skeletal muscle tremors by modulating/damping contractile oscillations, e.g., by introducing or administering a therapeutic as described above. The present invention is not limited to treating cardiac arrhythmias and skeletal muscle tremors and includes treating or modulating other conditions with undesirable contractile oscillations.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows coupled vs. uncoupled spontaneous auto-oscillatory contractions (SPOC). A twitch contraction typical of cardiac muscle is depicted as an increase and decrease in force (black line) coupled to a corresponding increase and decrease in [Ca²⁺] (left figure). Uncoupled (oscillatory) twitch contractions (black lines) occur following the loss of MyBP-C or cMyBP-C phosphorylation and are shown as multiple cycles of contraction and relaxation that occur in the presence of a constant level of Ca²⁺. Note that during cycles of oscillatory SPOC contractions, relaxation occurs without a concomitant decrease in [Ca²⁺].

FIG. 2 shows the proposed impact of SPOC on health and disease. Potentially adaptive responses of SPOC include faster, more efficient contractile kinetics, enhanced mechano-electrical feedback, and more coordinated contraction and relaxation across multiple cells or cardiac regions. Conversely, SPOC dysregulation may occur under conditions that chronically affect MyBP-C phosphorylation or reduce cMyBP-C expression, such as haploinsufficiency in HCM patients or proteolytic degradation of cMyBP-C during cardiac stress. In addition, dysregulation may include negative impacts on contractile properties and efficiency, deranged mechanoelectrical feedback that disrupts Ca²⁺ signaling or AP characteristics (e.g., delayed after depolarizations, early after depolarizations, slowed repolarization, etc.), and regional foci of hyper- or hypo-excitability.

FIGS. 3A and 3B show a cut-and-paste approach for the removal and replacement of cMyBP-C N′-terminal domains in cardiomyocytes from homozygous Spy-C mice. FIG. 3A shows a gene-edited Spy-C mouse express modified cMyBP-C with a TEV protease recognition site and a SpyTag inserted between domains C7 and C8. Inset. Immunofluorescence localization of cMyBP-C in homozygous (HO) and wild-type (WT) Spy-C myocytes showing expected localization in each ½ sarcomere. Z-lines are shown stained with a-actinin (light gray/white). FIG. 3B shows a cut and paste of cMyBP-C in Triton X-100 permeabilized cardiomyocytes from HO Spy-C mice. (1) CUT: TEVp treatment releases genetically encoded (g) C0-C7 domains which are soluble and can be removed from sarcomeres through gentle rinsing. Inset. Immunofluorescence shows loss of cMyBP-C stripes after TEVp treatment in HO myocytes but not WT myocytes. (2) PASTE: New recombinant rC0-C7-sc domains (encoding any desired modification such as mutations, deletions, fluorescent probes) are added to the bath where they become covalently attached to st-C8-C10 on the thick filament via a spontaneous bond formed between SpyCatcher (sc) and SpyTag (st). Inset. Immunofluorescence localization showing reappearance of cMyBP-C stripes after addition of rC0-C7-sc in HO myocytes but not rC0-C7 lacking encoded SpyCatcher. Other embodiments of the cut-and-paste approach are described in U.S. Pat. No. 11,242,368, which is incorporated herein by reference in its entirety.

FIG. 4 shows a representative western blot showing relative cMyBP-C expression (top, left panel) in triplicate samples from the left atrium (LA) and left ventricle (LV) from a cat heart. Actin (bottom, left panel) is shown as a loading control. The right panel shows summary data of normalized LV/LA ratios in mouse and cat hearts, indicating greater expression of cMyBP-C in ventricles compared to atria in both species.

### DETAILED DESCRIPTION OF THE INVENTION

As previously discussed, the present invention provides methods and compositions for modulating, e.g., damping, contractile oscillations. As an example, the present invention provides methods and compositions for modulating, e.g., damping, contractile oscillations by targeting and/or mimicking myosin binding protein C (MyBP-C) in muscles, e.g., heart and skeletal muscles. The present invention includes therapeutics (e.g., small molecules, peptides, enzymes, antibodies, oligonucleotides, drugs, compositions, physical stimuli, etc.) that regulate, e.g., dampen the oscillations. In certain embodiments, the therapeutics target MyBP-C or cMyBP-C. However, the present invention is not limited to targeting MyBP-C or cMyBP-C and includes targeting other molecules involved in the contractile oscillation process. As a non-limiting example, it may be determined that myosin is an appropriate target; other targets may be found to include protein kinase A (PKA) or other signaling molecules involved in MyBP-C's (or cMyBP-C) function, molecules or pathways involved in muscle lattice spacing, etc.

The present invention features compositions for modulating contractile oscillations in the heart, which target and/or mimic cardiac myosin binding protein C (cMyBP-C). The present invention may also feature compositions for modulating contractile oscillations in skeletal muscle, which target and/or mimic myosin-binding protein C (MyBP-C).

In some embodiments, the composition may target and/or mimic at least a portion of the N-terminal domain of cMyBP-C or MyBP-C. The compositions described herein may modulate, e.g., damp, contractile oscillations. In some embodiments, the composition is a small molecule, peptide, antibody, oligonucleotide, or a combination thereof.

In some embodiments, the present invention features a composition for modulating contractile oscillations in the heart, the composition comprising a small molecule. The composition may target and/or mimic cardiac myosin-binding protein C (cMyBP-C) or a portion thereof. In other embodiments, the present invention features a composition for modulating contractile oscillations in skeletal muscle, the composition comprising a small molecule. In some embodiments, the composition comprising a small molecule may target and/or mimic myosin-binding protein C (MyBP-C) or a portion thereof. In some embodiments, the composition comprising a small molecule targets and/or mimics the N-terminal domain of cMyBP-C or MyBP-C or a portion thereof.

In some embodiments, the present invention features a composition for treating a cardiac arrhythmia and/or skeletal muscle tremors, said composition comprising a small molecule that damps spontaneous auto-oscillatory contraction (SPOC) by converting sarcomere auto-oscillations to coordinated contractions.

In some embodiments, the present invention features a composition for modulating contractile oscillations in the heart, the composition comprising a peptide. The composition may target and/or mimic cardiac myosin-binding protein C (cMyBP-C) or a portion thereof. In other embodiments, the present invention features a composition for modulating contractile oscillations in skeletal muscle, the composition comprising a peptide. The composition comprising a peptide may target and/or mimic myosin-binding protein C (MyBP-C) or a portion thereof. In some embodiments, the composition comprising a peptide targets and/or mimics the N-terminal domain of cMyBP-C or MyBP-C or a portion thereof.

In some embodiments, the peptide comprises at least a portion of the cMyBP-C or MyBP-C. In some embodiments, the peptide comprises at least a portion of the N-terminal domain of the cMyBP-C or MyBP-C. In some embodiments, the peptide comprises at least a portion of the M-domain of the cMyBP-C or MyBP-C.

In some embodiments, the present invention features a composition for treating a cardiac arrhythmia and/or skeletal muscle tremors, said composition comprising a peptide that damps spontaneous auto-oscillatory contraction (SPOC) by converting sarcomere auto-oscillations to coordinated contractions.

In some embodiments, the present invention features a composition for modulating contractile oscillations in the heart, the composition comprising an enzyme. The composition may target cardiac myosin-binding protein C (cMyBP-C). In other embodiments, the present invention features a composition for modulating contractile oscillations in skeletal muscle, the composition comprising an enzyme. The composition may target myosin-binding protein C (MyBP-C). In some embodiments, the composition comprising an enzyme targets the N-terminal domain of cMyBP-C or MyBP-C.

In some embodiments, the enzyme comprises a phosphatase enzyme. In some embodiments, the enzyme targets (e.g., dephosphorylates) cMyBP-C or MyBP-C.

In some embodiments, the present invention features a composition for treating a cardiac arrhythmia and/or skeletal muscle tremors, said composition comprising an enzyme that damps spontaneous auto-oscillatory contraction (SPOC) by converting sarcomere auto-oscillations to coordinated contractions.

In some embodiments, the present invention features a composition for modulating contractile oscillations in the heart, the composition comprising an oligonucleotide. The composition may target and/or mimic cardiac myosin-binding protein C (cMyBP-C) or a portion thereof. In other embodiments, the present invention features a composition for modulating contractile oscillations in skeletal muscle, the composition comprising an oligonucleotide. The composition may target and/or mimic myosin-binding protein C (MyBP-C) or a portion thereof. In some embodiments, the composition comprising the oligonucleotide targets or mimics the N-terminal domain of cMyBP-C or MyBP-C or a portion thereof.

In some embodiments, the present invention features a composition for treating a cardiac arrhythmia and/or skeletal muscle tremors, said composition comprising an oligonucleotide that damps spontaneous auto-oscillatory contraction (SPOC) by converting sarcomere auto-oscillations to coordinated contractions.

The present invention features a composition for treating cardiac arrhythmia and/or skeletal muscle tremors, said composition comprising a therapeutic that damps spontaneous auto-oscillatory contraction (SPOC) by converting sarcomere auto-oscillations to coordinated contractions.

The present invention also features compositions (e.g., small molecule, peptide, antibody, oligonucleotide, or a combination thereof) for use in a method for the treatment of cardiac arrhythmia and/or skeletal muscle tremors, said composition comprising a therapeutic that damps spontaneous auto-oscillatory contraction (SPOC) by converting sarcomere auto-oscillations to coordinated contractions.

The present invention may also feature a method of treating cardiac arrhythmias and/or skeletal muscle tremors in a patient in need thereof, said method comprising administering to the patient a composition that targets or mimics cMyBP-C and/or MyBP-C.

The present invention also includes methods of treating cardiac arrhythmias and/or skeletal muscle tremors by modulating/damping contractile oscillations, e.g., by introducing or administering a therapeutic as described above. In some embodiments, the therapeutic dampens spontaneous auto-oscillatory contraction (SPOC) by converting sarcomere auto-oscillations to coordinated contractions. The present invention is not limited to treating cardiac arrhythmias and skeletal muscle tremors and includes treating or modulating other conditions with undesirable contractile oscillations.

Without wishing to limit the present invention to any theory or mechanism, it is believed that abnormalities with respect to cMyBP-C(e.g., decrease in cMyBP-C, misregulation of cMyBP-C, etc.) results in sarcomeres being more susceptible to oscillatory behaviors.

Without wishing to limit the present invention to any theory or mechanism, it is believed that detecting MyBP-C in blood (which may be associated with a heart attack or cardiac stress, atrial fibrillation, hypertrophic cardiomyopathies, exercise, etc.) may be an indication of a MyBP-C(e.g., cMyBP-C) abnormality and/or an increase in oscillatory behavior in sarcomeres.

### EXAMPLE

The following is a non-limiting example of the present invention. It is to be understood that said example is not intended to limit the present invention in any way. Equivalents or substitutes are within the scope of the present invention.

Excitation-contraction (EC) coupling, the process whereby a single membrane action potential causes the release of intracellular Ca²⁺ from the sarcoplasmic reticulum, and the released Ca²⁺ activates a single twitch contraction, is central dogma in striated muscle contraction (i.e., heart and skeletal muscles). However, the present invention challenges this dogma by showing that selective loss of cardiac myosin binding protein-C(cMyBP-C), a regulatory protein of cardiac muscle sarcomeres, can uncouple the one-to-one relationship of excitation-contraction in detergent-permeabilized cardiomyocytes so that multiple cycles of contraction and relaxation occur continuously when sarcomeres lacking cMyBP-C are activated by constant Ca²⁺. This behavior is known as spontaneous auto-oscillatory contraction (SPOC). Until now, a physiological role for SPOC has not been known. Based on Inventor's discovery that either loss of cMyBP-C or cMyBP-C phosphorylation induces SPOC in cardiomyocytes, the present invention proposes that auto-oscillatory contractions are a normal fundamental property of cardiac muscle sarcomeres that is regulated by cMyBP-C and possibly by other factors that directly affect sarcomere properties, e.g., that cMyBP-C damps sarcomere oscillations in a phosphorylation-dependent way by inhibiting relaxation in the presence of Ca²⁺.

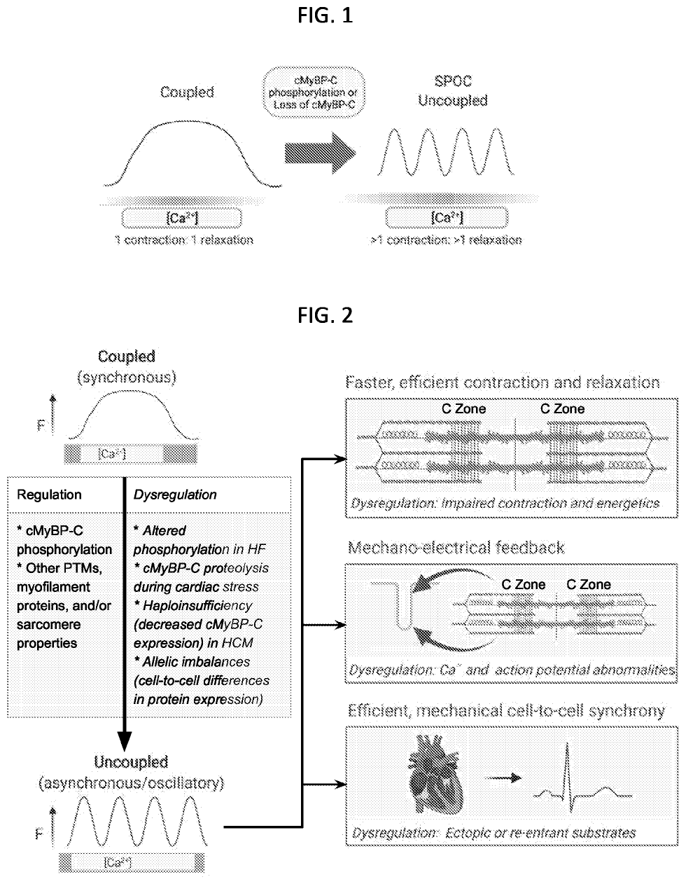

The present invention proposes that cMyBP-C plays a role in coupling relaxation to a decrease in Ca²⁺ since loss of cMyBP-C allows relaxation to occur despite constant activating Ca²⁺ (see FIG. 1). The present invention also proposes that dysregulation of SPOC can disrupt normal excitation-contraction coupling and directly cause arrhythmias through a reverse process, “contraction-excitation coupling”, where uncoordinated mechanical activity influences cell electrical signaling. Thus, the present invention provides methods and compositions for treating cardiac arrhythmias with drugs that dampen SPOC by converting sarcomere auto-oscillations to coordinated contractions.

The idea that discrete damping factors exist in vertebrate muscles that limit sarcomere auto oscillations has been largely overlooked. Inventor's discovery using an exclusive “cut and paste” approach that permits selective removal and replacement of cMyBP-C from cardiac sarcomeres showed that acute loss of cMyBP-C induces vigorous contractile oscillations (SPOC). Without wishing to limit the present invention to any theory or mechanism, it is believed that MyBP-C is one of the damping factors because phosphorylation of cMyBP-C did not fully damp the auto oscillations. The present invention proposes that sarcomere auto-oscillations are a regulated property of muscle contraction.

The idea that sarcomeres are capable of intrinsic auto-oscillatory contractions (i.e., recurring cycles of contraction and relaxation that occur independently of changes in intracellular Ca²⁺) has far reaching implications for normal striated muscle behavior and dysfunction during disease, e.g., the potentially adaptive significance of oscillatory contractions induced under β-adrenergic stimulation for increased inotropy and lusitropy in cardiac muscle (see FIG. 2). For instance, the process of moving Ca²⁺ into and out of the cytosol for each contraction is both time-consuming and energetically expensive so mechanical (stretch) activation was thought to have evolved in insect flight muscles (IFM) to achieve high frequency contraction while minimizing the high energetic costs associated with Ca²⁺ transport during each wingbeat. Similar considerations may apply to heart or skeletal muscles where auto-oscillatory sarcomere contractions could allow for greater contraction and relaxation speeds at improved energetic efficiency. Indeed, reduced energetic efficiency is thought to be a major factor contributing to or exacerbating disease etiologies such as hypertrophic cardiomyopathies and heart failure. Other potential advantages of SPOC include the ability of oscillations to propagate rapidly across multiple sarcomeres and across multiple cells as SPOC waves travel in coherent patterns. Such coordinated inter-sarcomere and multicellular activity may be energy sparing, especially in the heart under conditions of the increased inotropic and lusitropic drive where coordination across cardiac regions could augment the heart's conduction system to produce more powerful and coordinated heartbeats. Dysregulation of SPOC may also cause or exacerbate the clinical course of diseases involving cMyBP-C because cMyBP-C protein expression and phosphorylation are commonly reduced in cardiac diseases. For instance, mutations in MYBPC3, the gene encoding cMyBP-C, are among the most frequent causes of hypertrophic cardiomyopathy (HCM), where most mutations lead to reduced cMyBP-C protein expression (haploinsufficiency). Cardiac stress also results in proteolytic cleavage of cMyBP-C at a calpain site located within the M-domain. Therefore, cMyBP-C haploinsufficiency in HCM patients or in response to cardiac stress and/or during natural aging could result in increased SPOC and dysregulation of myofilament relaxation similar to that seen in Spy-C myocytes following TEVp treatment. Chronic dysregulation of SPOC activity could then potentially alter contractile kinetics, energetics, excitation/contraction coupling, or Ca²⁺ homeostasis. Allelic and cell-to-cell imbalances in cMyBP-C expression in myocytes of HCM patients could further exacerbate dysregulation leading to changes in source-sink dynamics between neighboring cells that create ectopic or reentry foci for arrhythmia. The opposite effects, i.e., reduced SPOC and slowed contraction and relaxation kinetics may occur if cMyBP-C is chronically hypo-phosphorylated as in heart failure patients.

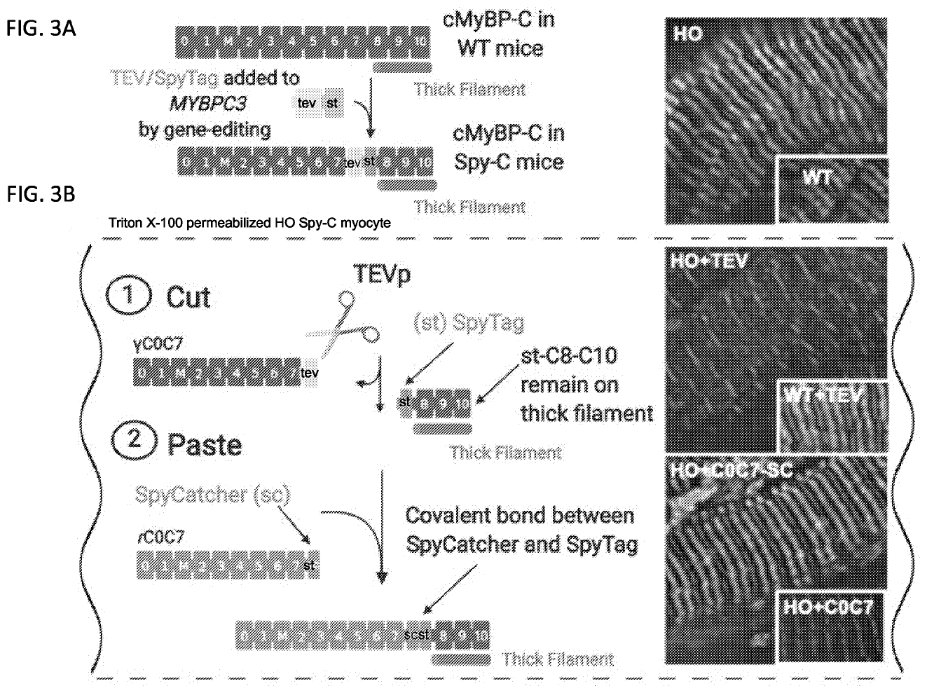

The belief that SPOC is a normal, intrinsic feature of muscle sarcomeres and that it is a regulated property that contributes to muscle function is conceptually innovative. Although, Sasaki et al. noted that SPOC frequency correlated with heart rate in mammals, the idea that SPOC is a regulated variable that contributes to muscle function has not been previously explored. Second, the idea that some arrhythmias may have purely myogenic origins, i.e., due to a primary myofilament dysfunction (apart from dysfunction in myocyte electrical signaling or substrate fibrosis), is conceptually innovative in that it challenges the central dogma of excitation-contraction coupling where cycles of contraction and relaxation are exclusively driven by electrical and chemical signaling upstream of sarcomeric contraction. This idea has immediate translational impact by suggesting new therapeutic targets for the treatment of arrhythmias such as treatment with cMyBP-C N′-terminal domains to damp SPOC and coordinate contraction in heart. Third, the approach for systematically studying SPOC in permeabilized myocytes from Spy-C mice and in Spy-C mice treated with AAV TEV protease is innovative and relies on the novel “cut and paste” approach developed exclusively by Inventor. The approach allows cMyBP-C to be selectively removed and replaced from its native position in sarcomeres in situ within minutes (see FIGS. 3A and 3B). The method thus fills a methodological gap between in vitro studies where there is little to no spatial or temporal control over cMyBP-C interactions with its targets and in vivo transgenic animal models that preserve cMyBP-C localization but that are costly and time consuming to generate and that may have confounding secondary effects due to cardiac remodeling.

The present invention describes gene-edited Spy-C mice developed exclusively by Inventor to allow manipulation of MyBP-C in contracting sarcomeres and in hearts of gene-edited Spy-C mice in vivo. Additional SpyC1 and SnoopC2 gene-edited mice allow manipulation in slow and fast twitch skeletal muscles, respectively. An overview of the “cut and paste” approach is shown in FIGS. 3A and 3B.

Without wishing to limit the present invention to any theory or mechanism, it is believed that the regulatory M-domain of cMyBP-C is required to damp contractile auto-oscillations through inhibition of myosin cross-bridge relaxation. Preliminary data from Inventor suggests that the phosphorylatable M-domain of cMyBP-C is required to dampen SPOC and that domains C0-C2 (inclusive of the M-domain) are sufficient to inhibit SPOC. FIG. 1 shows that the primary difference between coupled and auto-oscillatory twitch contractions (i.e., SPOC) is that relaxation is uncoupled from a decrease in Ca²⁺ so that relaxation in SPOC occurs despite the presence of [Ca²⁺]. The present invention describes testing the effects of recombinant domains with or without phosphorylation at each of the 3 canonical phosphorylation sites for their ability to damp SPOC in detergent-permeabilized myocytes and for their effects on relaxation in myofibrils. Force measurements in myofibrils allow for measuring the two phases (fast and slow) of relaxation, where the first (slow) phase is limited by cross-bridge detachment from the thin filament. The faster, more chaotic phase occurs when cross-bridge detachment proceeds to the point that sarcomeres suddenly “yield” and rapidly re-lengthen. MyBP-C could thus slow relaxation by affecting either or both phases.

Experiments in permeabilized myocytes include testing effects of full-length replacement of cMyBP-C(i.e., domains C0-C7-sc) versus replacement with selected N′-terminal domains (e.g., C0-C2-sc) with and without phosphorylation of the M-domain to determine domains that are necessary and sufficient to affect SPOC. Additional experiments investigate the effects of N terminal domains without SpyCatcher (because SpyCatcher is needed for covalent localization of cMyBP-C on the thick filament by covalent ligation to SpyTag in gene-edited cMyBP-C, see FIGS. 3A and 3B) to determine whether binding of cMyBP-C N′-terminal domains to the thin filament alone is sufficient to damp SPOC or whether cMyBP-C needs to be properly localized at its endogenous position in sarcomeres to damp SPOC. Additional experiments include effects of C0-C2-sc in the presence of dextran to compress thick and thin filament lattice spacing to determine if C0-C2-sc damps SPOC by limiting lattice expansion during contraction. Effects of myosin activators (omecamtiv, mecarbil) and inhibitors (mavacamten, blebbistatin) are investigated to determine whether drugs that specifically target myosin can also dampen SPOC by affecting myosin crossbridge kinetics.

Without wishing to limit the present invention to any theory or mechanism, it is believed that MyBP-C dampens SPOC by delaying the fast, chaotic phase of relaxation, and this phase of relaxation may be accelerated following loss of cMyBP-C C0-C7 domains (after TEVp treatment) and subsequently slowed after ligation with C0-C7-sc or C0-C2-sc.

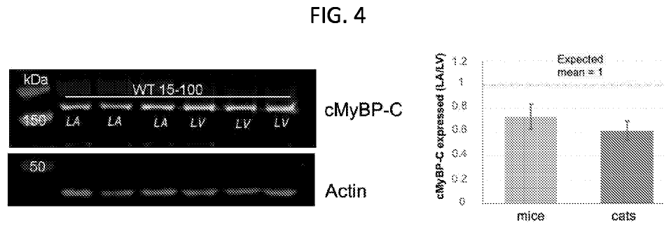

Without wishing to limit the present invention to any theory or mechanism, it is believed that reduced cMyBP-C expression causes sarcomere auto-oscillations (SPOC) and initiates myofilament generated cardiac arrhythmias in vivo. Mutations in cMyBP-C (MYBPC3) are the most common cause of hypertrophic cardiomyopathy (HCM), a disease affecting 1:250-500 people and a common cause of sudden cardiac death. HCM is widely recognized as a “disease of the sarcomere” because most mutations occur in myofilament contractile or regulatory proteins, but it has remained enigmatic how primary defects in contractile proteins can cause arrhythmias. Prevailing ideas suggest that mutations in contractile proteins indirectly cause excitation or signaling disturbances via secondary substrate remodeling (e.g., myocyte disarray or fibrosis) that subsequently lead to arrhythmogenic conduction defects. In contrast, the present invention proposes that loss of cMyBP-C and its ability to synchronize contraction across sarcomeres and cells directly causes chaotic contractions typical of atrial or ventricular fibrillations via dysregulation of SPOC. Preliminary data from Inventor further shows that atrial myocytes express ˜40% less cMyBP-C than ventricular myocytes in mouse and cat species (see FIG. 4), suggesting that atrial myocytes may be especially predisposed to fibrillation upon loss of cMyBP-C as might occur in disease or due to aging in people. The present invention proposes that dysregulation of SPOC is a previously unrecognized etiology in cardiac arrhythmogenesis, especially atrial fibrillation (AF). Because AF is the most common clinical cardiac arrhythmia, affecting over 6 million people per year in the US alone and is associated with increased risk of heart failure, stroke, and mortality, targeting cMyBP-C or otherwise inhibiting SPOC via myosin inhibitors or activators has the potential for new treatment options for AF patients.

TEV protease (TEVp) is delivered via AAV9 to hearts of wild-type (control) and homozygous Spy-C mice (3-4 days old, pericardial injection) to produce rapid knockout of cMyBP-C C0-C7 domains in Spy-C mice. AAV9 constructs and the cardiac troponin TNT4 promoter are used to direct expression preferentially to both atrial and ventricular cardiomyocytes. mCherry encoded upstream of TEVp are used to confirm protein expression using immunofluorescence in tissue sections and by western blotting for TEVp and cleaved cMyBP-C in tissue lysates. Standard histological staining methods are used to assess fibrosis in atria and ventricles. Male and female mice are phenotyped initially at 4, 8, 12, and 16 weeks post injection by echocardiography to determine cardiac function and contractile effects due to loss of cMyBP-C. Susceptibility to arrhythmia is measured in ambulatory conscious mice (males and females) using implantable telemetry (DSI, HD-X11 transmitter) to obtain ECG rhythm strips. Programmed electrical stimulation in instrumented anesthetized mice (males and females) is used to evaluate susceptibility to induced monomorphic ventricular tachycardia (mmVT). Without wishing to limit the present invention to any theory or mechanism, it is believed that knockdown of cMyBP-C using viral delivery of TEVp will result in increased SPOC and increased susceptibility to arrhythmias in both ambulatory (unprovoked) and instrumented (provoked) mice. Embodiments of the present invention can be freely combined with each other if they are not mutually exclusive.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A composition for modulating contractile oscillations in the heart, the composition targets or mimics cardiac myosin binding protein C (cMyBP-C).

2. The composition of claim 1, wherein the composition targets the N-terminal domain of cMyBP-C.

3. The composition of claim 1, wherein the composition for modulating contractile oscillations dampens contractile oscillations.

4. The composition of claim 1, wherein the composition is a small molecule, peptide, antibody, oligonucleotide, or a combination thereof.

5. A composition for modulating contractile oscillations in skeletal muscle, the composition targets or mimics myosin binding protein C (MyBP-C).

6. The composition of claim 5, wherein the composition targets the N-terminal domain of MyBP-C.

7. The composition of claim 5, wherein the composition for modulating contractile oscillations dampens contractile oscillations.

8. The composition of claim 5, wherein the composition is a small molecule, peptide, antibody, oligonucleotide, or a combination thereof.

9. (canceled)

10. A method for treating a cardiac arrhythmia, said method comprising administering a therapeutic composition that dampens spontaneous auto-oscillatory contraction (SPOC) by converting sarcomere auto-oscillations to coordinated contractions.

11. The method of claim 11, wherein the therapeutic composition targets or mimics cardiac myosin binding protein C (cMyBP-C).

12. The method of claim 11, wherein the therapeutic composition that targets or mimics cardiac myosin binding protein C (cMyBP-C) is a small molecule, peptide, antibody, oligonucleotide, or a combination thereof.

13-34. (canceled)

35. The composition of claim 3, wherein the contractile oscillations comprise spontaneous auto-oscillatory contraction (SPOC).

36. The composition of claim 7, wherein the contractile oscillations comprise spontaneous auto-oscillatory contraction (SPOC).
